(19) United States
(12) Patent Application Publication
(10) Pub. No.: US 2016/0029996 A1
(43) Pub. Date: Feb. 4, 2016

(54) DEVICE AND METHOD FOR USING IVUS DATA TO CHARACTERIZE AND EVALUATE A VASCULAR GRAFT CONDITION

(71) Applicants: Eric JOHNSON, Woodside, CA (US); Jeremy STIGALL, Carlsbad, CA (US)

(72) Inventors: Eric JOHNSON, Woodside, CA (US); Jeremy STIGALL, Carlsbad, CA (US)

(21) Appl. No.: 14/814,069

(22) Filed: Jul. 30, 2015

Related U.S. Application Data
(60) Provisional application No. 62/032,431, filed on Aug. 1, 2014.

(51) Int. Cl. A61B 8/06; A61B 8/00; A61F 2/06; A61B 8/12; A61B 8/08

(52) U.S. Cl. CPC A61B 8/06; A61B 8/12; A61B 8/5246; A61F 2/06; A61B 8/461

(57) ABSTRACT

There are described systems and methods of collecting ultrasound information related to a vascular graft condition of a patient and evaluation of the information using a virtual histology tree and hemodynamic information.

### CROSS REFERENCE TO RELATED APPLICATIONS

This application claims the benefit under 35 U.S.C. 119 of U.S. Patent Application No. 62/032,431, filed Aug. 1, 2015, titled “DEVICE AND METHOD FOR USING IVUS DATA TO CHARACTERIZE AND EVALUATE A VASCULAR GRAFT CONDITION”. This application is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### STATEMENT AS TO FEDERALLY SPONSORED RESEARCH

NONE

### DESCRIPTION OF RELATED ART

Intravascular ultrasound (“IVUS”) combined with a technique called virtual histology (“VH”) has been particularly successful in recognizing the morphology of plaque in vivo (i.e., the amount and composition of plaque in the patient's body). Numerous systems for detecting and characterizing using IVUS with VH have been developed including IVUS based techniques to characterize blood flow.

It would also be desirable to find applications where the benefits of IVUS techniques may be used in vascular graft applications.

### FIELD

Embodiments of the invention relate to improved devices and methods for intraluminal or intravascular identification evaluation and or monitoring of patients based on an evaluation of physiological or other parameters related to one or more vascular graft or stent characteristics.

### SUMMARY OF THE DISCLOSURE

In general, in one embodiment, a method for assessing a hemodynamic condition in a patient having an implanted vascular graft, including advancing an IVUS capable catheter through a lumen within or adjacent to the implanted vascular graft or a portion of the vascular graft; collecting intraluminal ultrasound information related to one or more hemodynamic characteristics of a flow within the lumen or the portion of the vascular graft; and comparing the information from the collecting step to information related to a desired hemodynamic characteristic of a lumen portion within or adjacent to an implanted vascular graft or within a portion of an implanted vascular graft using an image comparison algorithm; and providing a result of the comparing step.

This and other embodiments can include one or more of the following features. In one aspect, the collecting step can be performed periodically after implantation of the vascular graft. In another aspect, in any of the methods above, the vascular graft can be an arterial bypass graft.

In a further aspect, in any of the techniques above, the vascular graft can be an arteriovenous graft. In an alternative aspect, the steps of advancing and collecting can be performed in a blood vessel adjacent, proximate to or joined to one or more portions of the arterial bypass graft. In yet another aspect, the steps of advancing and collecting can be performed in a blood vessel adjacent, proximate to or joined to one or more portions of the arteriovenous graft. In still another aspect, in any of the methods above, the one or more hemodynamic characteristics can include one or more of wall shear stress, an indication of turbulent flow, an indication of laminar flow, an indication of blood velocity, and an indication of one or more blood velocity patterns. In one aspect, in any of the methods above, when the advancing and the collecting step are performed a transducer or a catheter can be moved from one or more of from an artery through a graft to another artery or the same artery; (ii) from an artery through a graft to a vein; (iii) from a vein through a graft to the same or another vein; (iv) from a vein through a graft to an artery. In another aspect, any of the techniques above can further include moving an IVUS transducer or catheter with or against the flow in an artery or vein or in a direction towards the heart or in a direction away from the heart or in a direction with the prevailing or intended flow or in a direction against the prevailing or intended flow.

In general, in one embodiment, a method for assessing a condition of a tissue within or adjacent to an implanted vascular graft includes advancing an IVUS capable catheter through a vascular lumen within or adjacent the implanted vascular graft; collecting intraluminal ultrasound information related to a composition of a portion of the vascular lumen or vascular graft; and comparing the information from the collecting step to information in a virtual histology tree related to the composition of the portion of the vascular lumen or the vascular graft using an image comparison algorithm; and providing a result of the comparing step.

This and other embodiments can include one or more of the following features. In one aspect, the collecting step can be performed after a time period has elapsed after implantation of the vascular graft. In another aspect, the collecting step can be performed periodically after implantation of the vascular graft. In a further aspect, the vascular graft can be an arterial bypass graft. In an alternative aspect, the vascular graft can be an arteriovenous graft. In yet another aspect, the steps of advancing and collecting can be performed in a blood vessel adjacent, proximate to or joined to one or more portions of the arterial bypass graft. In still another aspect, the steps of advancing and collecting can be performed in a blood vessel adjacent, proximate to or joined to one or more portions of the arteriovenous graft. In one aspect, the steps of advancing and collecting can be performed in a blood vessel adjacent, proximate to, along or within a fistula. In another aspect, in any of the methods above, the steps of advancing and collecting can be performed in a blood vessel adjacent, proximate to, along or within a fistula so as to obtain one or more hemodynamic characteristics and can include one or more of wall shear stress, an indication of turbulent flow, an indication of laminar flow, an indication of blood velocity, and an indication of one or more blood velocity patterns. In a further aspect, when the advancing and the collecting step can be performed a transducer or a catheter can be moved from one or more of from an artery through a graft to another artery or the same artery; (ii) from an artery through a graft to a vein; (iii) from a vein through a graft to the same or another vein; (iv) from a vein through a graft to an artery. In an alternative aspect, any of the methods above can further include moving an IVUS transducer or catheter with or against the flow in an artery or vein or in a direction towards the heart or in a direction away from the heart or in a direction with the prevailing or intended flow or in a direction against the prevailing or intended flow. In yet another aspect, any of the methods above can be performed serially or simultaneously with one or more steps of any of the techniques above.

In general, in one embodiment, a method for assessing an vascular graft condition in a patient, includes advancing an IVUS capable catheter through a lumen within or adjacent a site of an vascular graft condition in a patient; collecting transluminal or intraluminal ultrasound information on the vascular graft or other lesion or condition of the patient; and comparing the information from the collecting step to information in a virtual histology tree related to the vascular graft condition using an image comparison algorithm; and providing a result of the comparing step to a health care provider.

This and other embodiments can include one or more of the following features. In one aspect, the collecting step can be performed after the patient has received one or more treatments for a vascular graft condition. In another aspect, providing the result can include relative information about the size, composition, or position of a fistula or region of interest related to the vascular graft condition. In a further aspect, any of the methods above can further include collecting ultrasound information of a portion of a stent or graft to determine the location, position, orientation, configuration or amount or degree of deployment or one or more portions, components or elements of the stent or graft. In an alternative aspect, in any of the methods above the step of advancing an ultrasound probe or catheter can be performed with particular regard to one or more regions of interest for the configuration of the stent-graft under evaluation.

### BRIEF DESCRIPTION OF THE DRAWINGS

A better understanding of the features and advantages of the various embodiments of the present invention will be obtained by reference to the following detailed description that sets forth illustrative embodiments, in which the principles of the invention are utilized, and the accompanying drawings of which:

FIG. 1 is a schematic view of a tissue characterization system using IVUS and VH.

FIG. 2 is a side view of an arterial bypass stent placed through tissue track such that a proximal portion is in communication with a coronary artery and a distal portion is in communication with a coronary vein.

FIGS. 3A and 3B provide illustrative hemodynamic patterns within exemplary grafts depending upon graft location such as artery—vein (FIG. 3A) and artery—artery (FIG. 3B).

FIG. 4 is a flow chart of an illustrative method of assessing a vascular graft condition and obtaining data for use in evaluating imaging and hemodynamic information.

### DETAILED DESCRIPTION

In accordance with embodiments of this invention, a patient's physiological parameters are ascertained using information collected from one or more of intravascular ultrasound (IVUS), intraluminal ultrasound, transluminal ultrasound or external ultrasound, each alone or in any combination with any form of hemodynamic information obtained in any form alone or in combination with virtual histology (VH) of healthy or representative vascular graft tissue or fistula forming tissue and the like at or near the vascular graft or region related to the hemodynamic information. Thereafter, these physiological parameters are evaluated to predict whether the patient's current physiology (i.e., post vascular graft implantation) presents a progression of or diminution of a condition or absence or diminution of a state compared to prior data from that patient, a similarly situated patient or patients in an “at large” population. In another aspect, there is provided a vascular graft tissue evaluation database of vascular tissue and healthy fistula formation or maturation tissue useful in image based analysis to identify or evaluate the post vascular graft condition of the vascular of a patient having an implanted vascular graft.

In particular, in one embodiment of the invention, one or more of physiological parameters of healthy tissue composition in a lumen is provided as related to the growth or formation of a fistula, or the incorporation or degradation of all or a portion of a vascular graft or a graft—tissue boundary condition, fistula tissue composition, state of fistula formation, effectiveness of any therapy provided to aid or enhance fistula formation or preserve a fistula already formed or to prevent restenosis or intimal hyperplasia or fistula size, dimension or characteristics that are gathered, contained and or compared to previously obtained physiological parameters, target values and ranges collected in a VH database and/or hemodynamic database. One or more hemodynamic parameters or stent or graft information is collected according to one or more of the parameters described in or suited to fistula or stent graft assessment as disclosed in any of

U.S. Publication No. 2006/0106452 to Niermann, published May 18, 2006, titled “STENT HAVING TWIST CANCELLATION GEOMETERY;” U.S. Publication No. 2007/0100437 to Welborn et al., published May 3, 2007, titled “DRAINAGE STENT AND ASSOCIATED METHOD;” U.S. Publication No. 2008/0306580 to Jenson et al., published Dec. 11, 2008, titled “BLOOD ACCESS APPARATUS AND METHOD;” U.S. Publication No. 2009/0054966 TO Rudakov et al., published Feb. 26, 2009, titled “ENDOVASCULAR DEVICE WITH MEMBRANE;” U.S. Publication No. 2010/0010613 to Dorn, published Jan. 14, 2010, titled “ARTERIOVENOUS FISTULA;” U.S. Publication No. 2011/0046720 to Shalev et al., published Feb. 24, 2011, titled “APPARATUS AND METHOD FOR CREATING ARTERIOVENOUS FISTULAS;” U.S. Publication No. 2011/0166642 to Ehr et al., published Jul. 7, 2011, titled “STENT CONFIGURATIONS;” U.S. Publication No. 2011/0184347 to Mason, published Jul. 28, 2011, titled “VASCULAR ACCESS DEVICE;” U.S. Pat. No. 3,882,862 to Berend, issued May 13, 1975, titled “ARTERIOVENOUS SHUNT;” U.S. Pat. No. 6,746,464 to Makower, issued Jun. 8, 2004, titled “DEVICE, SYSTEM AND METHOD FOR INTERSTITIAL TRANSVASCULAR INTERVENTION;” U.S. Pat. No. 7,828,814 to Brenneman et al., issued Nov. 9, 2010, titled “DEVICE AND METHOD FOR ESTABLISHING AN ARTIFICIAL ARTERIO-VENOUS FISTULA;” “The Role of Shear Forces in Arterial Branching,” by M. Zamir, The Journal of General Physiology, Vol. 67, pgs. 213-222, 1976; “Prosthetic arteriovenous grafts for hemodialysis” by Jacob A. Akoh, The Journal of Vaxcular Access 2009; 10: 137-147; “Intravascular Ultrasound: Principles and Cerebrovascular Applications,” Zacharatos et al., AJNR 31, pgs. 586-597, April 2010; “Intimal hyperplasia and hemodynamic factors in arterial bypass and arteriovenous grafts: a review,” by Hiroaki Haruguchi et al., The Japanese Society for Artificial Organs, 6:227-235, 2003; “Improved patency of prosthetic arteriovenous grafts with an acute anastomotic angle and flow diffuser,” by Hakaim et al., Journal of Vascular Surgery, Vol. 37, No. 5, 1032-1035, May, 2003; “Hemodynamic Shear Stress and Its Role in Atherosclerosis,” JAMA, Vol. 282, No. 21, pgs. 2035-2042, Dec. 1, 1999; “Has the incidence of end-state renal disease in the USA and other countries stabilized?” by Paul W. Eggers, National Institute of Diabetes and Digestive and Kidney Diseases, 20:241-245, 2011; and “Bioresorbable polymeric stents: current status and future promise,” by Eberhart et al., J. Biomater. Sci. Polymer Edn, Vol. 14, No. 4, pp. 299-312 (2003), each of which is herein incorporated by reference in its entirety.

Intravascular ultrasound (“IVUS”) combined with a technique called virtual histology (“VH”) has been particularly successful in recognizing the morphology of plaque in vivo (i.e., the amount and composition of plaque in the patient's body). The following systems for detecting and characterizing using IVUS with VH are disclosed in International Publication No. WO 2011/038305, published Mar. 31, 2011, titled “DEVICE AND METHOD FOR DETERMINING THE LIKELIHOOD OF A PATIENT HAVING A CLINICAL EVENT OR A CLINICALLY SILENT EVENT BASED ON ASCERTAINED PHYSIOLOGICAL PARAMETERS;” U.S. Pat. No. 5,724,978, Tenhoff, issued Mar. 10, 1998, titled “ENHANCED ACCURACY OF THREE-DIMENSIONAL INTRALUMINAL ULTRASOUND (ILUS) IMAGE RECONSTRUCTION,” U.S. Pat. No. 6,200,268, to Vince et al., issued Mar. 13, 2001, titled “VASCULAR PLAQUE CHARACTERIZATION;” U.S. Pat. No. 6,381,350, to Klingensmith et al., issued Apr. 30, 2002, titled “INTRAVASCULAR ULTRASONIC ANALYSIS USING ACTIVE CONTOUR METHOD AND SYSTEM;” U.S. Pat. No. 7,074,188 to Nair et al., issued Jul. 11, 2006, titled “SYSTEM AND METHOD OF CHARACTERIZING VASCULAR TISSUE;” U.S. Pat. No. 7,175,597 to Vince et al., issued Feb. 13, 2007, titled “NON-INVASIVE TISSUE CHARACTERIZATION SYSTEM AND METHOD;” U.S. Pat. No. 7,215,802 to Klingensmith et al., issued May 8, 2007, titled “SYSTEM AND METHOD FOR VASCULAR BORDER DETECTION;” U.S. Pat. No. 7,359,554 to Klingensmith et al., issued Apr. 15, 2008, titled “SYSTEM AND METHOD FOR IDENTIFYING A VASCULAR BORDER;” and U.S. Pat. No. 7,463,759 to Klingensmith et al., issued Dec. 9, 2008, titled “SYSTEM AND METHOD FOR VASCULAR BORDER DETECTION,” each of which are herein incorporated by reference in its entirety. An example of an IVUS system is the s5i™ Imaging System sold by Volcano Corporation of San Diego, Calif. Examples of OCT imaging systems include, but are not limited to, those disclosed in U.S. Publication No. 2007/0106155 to Goodnow et al., published May 10, 2007, titled “SYSTEM AND METHOD FOR REDUCING ANGULAR GEOMETRIC DISTORTION IN AN IMAGING DEVICE;” U.S. Publication No. 2008/0119701 to Milner et al., published May 22, 2008, titled “ANALYTE SENSOR METHOD AND APPARATUS;” U.S. Publication No. 2008/0287801 to Magnin et al., published Nov. 20, 2008, titled “IMAGING DEVICE IMAGING SYSTEM AND METHODS OF IMAGING;” U.S. Publication No. 2009/0018393, to Dick et al., published Jan. 15, 2009, titled “CATHETER FOR IN VIVO IMAGING,” U.S. Publication No. 2009/0046295 to Kemp et al., filed Feb. 19, 2009, titled “APPARATUS AND METHODS FOR UNIFORM SAMPLE CLOCKING;” U.S. Publication No. 2009/0093980 to Kemp et al., published Apr. 9, 2009, titled “REAL TIME SD-OCT WITH DISTRIBUTED ACQUISITION AND PROCESSING;” U.S. Publication No. 2009/0284749 to Johnson et al., published Nov. 19, 2009, titled “OCT Combining Probes and Integrated Systems,” and WIPO Published Patent Application No. WO 2009/023635 to Condit et al, published Feb. 19, 2009, titled “FORWARD-IMAGING OPTICAL COHERENCE TOMOGRAPHY (OCT) SYSTEMS AND PROBE”, the collective teachings of which, in their entirety, are incorporated herein by reference.

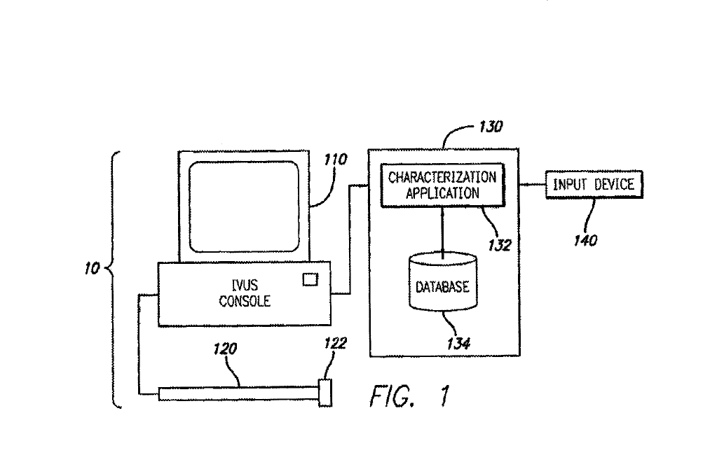

FIG. 1 illustrates a typical tissue-characterization system 10 using IVUS alone for hemodynamic parameter measurement and evaluation or in combination with VH (so called “VH-IVUS”) alone or in any combination with one or more mode of blood velocity indicating ultrasound methods, techniques or modes of assessing and analyzing intravascular ultrasound information. In the typical tissue—characterization system 10, an intra-vascular ultrasound (IVUS) console 110 is electrically connected to an IVUS catheter 120 and used to acquire RF backscattered data (i.e., IVUS data) from a blood vessel and/or one or more blood flow characteristics within a vessel or graft to gain information about the tissue or hemodynamic environment of the lumen or vascular graft or stent containing the IVUS catheter. The VH-IVUS console 110 typically includes a computing device 130 comprising a database 134 and a characterization application 132 electrically connected to the database 134 and adapted to receive IVUS data from the IVUS console 110 or directly from a transducer 122.

Specifically, a transducer 122 is attached to the end of the catheter 120 and is carefully maneuvered through a patient's lumen to a point of interest along the lumen before, after, within, proximate to, distal to or immediately adjacent to a vascular graft or portion of a vascular graft. The transducer is then pulsed to acquire signals reflected from the tissue of the lumen and any adjacent structure or object, such as a portion of a vascular graft. Because different types and densities of healthy tissue including growing tissue as in the growth related for the formulation of a fistula or the growth to absorb or cover a portion of a vascular graft absorb and reflect the ultrasound pulse differently, the reflected data (i.e., IVUS data) is used to image the lumen as well as surrounding structure or objects. In other words, the IVUS data can be used (e.g., by the IVUS console 110 or a separate computing device 130) to create an IVUS tissue image including hemodynamic information correlated to the position of the IVUS probe or location relative to an anatomical site or portion of an implanted vascular graft.

It should be appreciated that the IVUS console 110 depicted herein is not limited to any particular type of IVUS console, and includes all ultrasonic devices known to those skilled in the art (e.g., a Revolution® or EagleEye® IVUS catheter used in conjunction with an s5™ IVUS imaging system, all of which are sold by Volcano Corporation of San Diego, Calif.). It should further be appreciated that the IVUS catheter 120 depicted herein is not limited to any particular type of catheter, and includes all ultrasonic catheters known to those skilled in the art. Thus, for example, a catheter having a single transducer (e.g., adapted for rotation) or an array of transducers (e.g., circumferentially positioned around the catheter or longitudinally along the catheter 120) can be used with a graft-tissue characterization system 10.

It should be appreciated that the database 134 depicted herein includes, but is not limited to, RAM, cache memory, flash memory, magnetic disks, optical disks, removable disks, SCSI disks, IDE hard drives, tape drives and all other types of data storage devices (and combinations thereof, such as RAID devices) generally known to those skilled in the art. It should further be appreciated that the characterization application 132, as depicted and discussed herein, may exist as a single application or as multiple applications, locally and/or remotely stored. It should also be appreciated that the number and location of the components depicted in FIG. 1 do not limit a typical tissue—characterization system 10 but are merely provided to illustrate a typical tissue—characterization system 10. Thus, for example, a computing device 130 having a plurality of databases 134 or a remotely located characterization application 132 (either in part or in whole) or any combination of these may also be found in a typical tissue—characterization system 10.

In one embodiment of a typical tissue-graft or tissue graft hemodynamic characterization system 10, the characterization application 132 is adapted to receive and store characterization data (e.g., tissue type, vessel type, vessel-graft location, time since graft implantation, time since last fistula formation enhancement therapy, fistula development, prior or concurrent therapies, etc.). The characterization data may be determined prior to using the tissue-graft characterization system 10 or tissue-graft and hemodynamic characterization system as follows. After a specimen vascular object has been interrogated (e.g., IVUS data has been collected), a histology correlation is prepared. In other words, the specimen vascular object is dissected or cross-sectioned for histology. In one method of producing characterization data, the cross-section is previously marked, for example with a suture, so that the histology can be corresponded to a portion of the IVUS image. The cross-section is then prepared with a fixing and staining process that is well known in the art. The staining process allows a clinician to identify a tissue type(s), or a chemical(s) found within (e.g., a chemical corresponding to a particular tissue type, graft type etc.). The identified tissue type or types is then correlated to the IVUS data as will be explained below.

In one aspect, the systems and methods described herein utilize intraluminal ultrasound along with VH-IVUS techniques to predict, detect, evaluate or monitor changes in vascular physiology after graft implantation or therapy or recovery in order to monitor and/or prophylactically administer therapeutic or preventative care as part of the ongoing therapy to assist in tissue growth post implantation (i.e., to promote, assist or monitor fistula growth or maturation).

Currently, Virtual Histology classification trees are developed by collecting and correlating RF backscatter signal from an IVUS catheter with known histologic tissue types as disclosed in U.S. patent application Ser. No. 10/647,971, now U.S. Pat. No. 7,074,188, incorporated herein by reference. The RF data is transformed in to the frequency domain and the various power spectral characteristics of the backscattered signal are correlated with characterization data to determine signature parameters for each tissue type. However, these spectral characteristics of the tissue types vary for catheters operating at different frequencies and thus separate classification trees must be used. Accordingly, specific information about the catheter used for a clinical procedure is needed to select the appropriate classification tree for analyzing that data. For some vascular graft conditions, VH-IVUS imaging is quite practical because of suitable imaging resolution as well as intraluminal access for collection of transluminal and intraluminal images and data that may be collected and utilized for creation of vascular graft virtual histology trees.

The information from the catheter, at a minimum the operating frequency of the connected catheter 102, is used by the operation console 110 to select the appropriate VH classification tree for analyzing incoming IVUS data. In addition, information regarding specific catheter performance characteristics such as the unity gain value, the boot mode, the catheter sensitivity may also be stored on the catheter and used to further select the appropriate VH tree for analyzing data from the catheter imaging element. For example, it is envisioned that the operation console 110 could store VH classification trees for low, medium and high sensitivity catheters in each operating frequency. Then, based on information from the catheter regarding the operating frequency and sensitivity of the catheter, the operation console further tailor the selection of the appropriate VH classification tree. In an alternative embodiment, the VH classification tree for the specific catheter may be stored on the catheter. Here, when the catheter is placed in communication with the interface device, the catheter will relay the specific classification tree to use for analyzing the data it collects to the operation console and the operation console will simply download the classification tree from the catheter.

In various embodiments of the present invention, the tissue types and chemicals found within relate to those commonly found in various stages and types of vascular graft treatments.

In addition or alternatively, the VH database and/or Virtual Histology classification trees also includes correlation and comparison data for numerous healthy tissue-graft or tissue-stent boundary conditions. In one aspect, there is provided initial collection and evaluation to types of vascular graft implantation or fistula formation or tissue present in a stage of or expected stage of fistula formation. In one aspect, there is a method for an evaluation for comparison to initial evaluation in patient and compare to other patients at that stage of vascular graft implantation or fistula formation progression. In another aspect, a method provides an evaluation for comparison to evaluate or determine progression post therapy. In another aspect, the method allows for comparison to prior evaluation to determine progress post drug regime and evaluate effectiveness of drug regime. In one aspect, any one or more of the techniques described herein are used to establish a baseline set of patient specific data related to the treatment or disease to be treated. It is to be appreciated that any of the above may be combined, in any combination.

It should be appreciated that there may be many methods used to identify or characterize a cross-sectional object and the tissue beyond it as is well understood in the art besides the method just described. Thus, any identification/characterization method generally known to those skilled in the art may be used to characterize tissue both healthy and vascular graft implantation or fistula formation. The identified tissue type or characterization (i.e., characterization data) is then provided to the characterization application 132. In one embodiment, as shown in FIG. 1, the characterization data is provided via an input device 140 electrically connected to the computing device 130. The characterization data is preferably then stored in the database 134. It should be appreciated that the input device depicted herein includes, but is not limited to, a keyboard, a mouse, a scanner and all other data-gathering and/or data-entry devices generally known to those of skill. It should further be appreciated that the term tissue type or characterization, as these terms are used herein, include, but are not limited to, fibrous tissues, fibro-lipidic tissues, calcified necrotic tissues, calcific tissues, collagen compositions, cholesterol, thrombus, compositional structures (e.g., the lumen, the vessel wall, the medial-adventitial boundary, vessel-graft boundary, non-graft tissue composition, etc.) and all other identifiable characteristics used for the identification of vascular tissue or to discriminate fistula tissue from other tissue or between fistula tissues at one or more different stages of fistula formation progression as is generally known to those skilled in the vascular graft arts.

In one method of characterizing tissue, the characterization application 132 is adapted to create a VH image where specifically characterized tissue is associated with its corresponding region on an IVUS image. Specifically, digitized data is provided to the characterization application 132 (e.g., via the IVUS catheter 120) where the digitized data corresponds to the physiology of the patient's lumen and adjacent vascular graft implantation or fistula formation(s) and healthy tissue and fistula tissue. The data may be collected from blood vessels adjacent, proximate to or joined to one or more portions of a vascular graft. It is to be appreciated that the transducer 122 and/or catheter 120 may be advanced by way of example and not limitation, (i) from an artery through a graft to another artery or the same artery; (ii) from an artery through a graft to a vein; (iii) from a vein through a graft to the same or another vein; (iv) from a vein through a graft to an artery and in any of the above may advance with or against the flow in an artery or vein or in a direction towards the heart or in a direction away from the heart or in a direction with the prevailing or intended flow or in a direction against the prevailing or intended flow.

Although venography has been a preferred method for selecting sites having significant stenosis or flow disruption, ultrasonography, including duplex ultrasonography, could also be used in the alternative or in addition to identify obstructed, partially obstructed or for the measurement of flow within or adjacent to a stent or a graft site. In general, ultrasonography incorporates two elements: 1) Grayscale Ultrasound (e.g., from an IVUS imaging system 2) is used to visualize the structure or architecture of the involved lumen or involved stent or graft to identify characteristics of the lumen the stent or the graft such as, in the case of a lumen for example, stenoses (cross-sectional narrowing of a lumen); 2) Color-Doppler ultrasound imaging (e.g., from Volcano Corporation) is then used to visualize the flow or movement of a blood within the vein as well as other hemodynamic properties described herein or one or more responses to the hemodynamic environment by a lumen or structure such as a fistula; and typically presents both displays on the same screen (“duplex”) to facilitate interpretation. Ultrasonography can also be enhanced by tissue characterization such as the virtual histology characterization described herein, for example, as part of the s5i™ Imaging System with VH capability sold by Volcano Corporation of San Diego, Calif.

In addition or alternatively along with venography and ultrasonography, transcutaneous echography may also be applied to an accessible section of the lumen, the stent or the graft that may then be used to identify narrowing, obstruction sites and to confirm or exclude a significant stenosis or flow disruption at one or more sites adjacent to or within a stent or graft. Additional details for the use of one or more aspects of ultrasonography including flow information and imaging information may be obtain with reference to U.S. Pat. No. 8,289,284 to Glynn et al., issued Oct. 16, 2012, titled “CONTROLLER USER INTERFACE FOR A CATHETER LAB INTRAVASCULAR ULTRASOUND SYSTEM;” U.S. Pat. No. 8,529,506 to Brown et al., issued Sep. 10, 2013, titled “THERAPEUTIC DELIVERY DEVICES, SYSTEMS, AND METHODS FOR MULTIPLE SCLEROSIS, DEEP VEIN THROMBOSIS, AND PULMONARY EMBOLISM;” U.S. Publication No. 2009/0195514 to Glynn et al., published Aug. 6, 2009, titled “CONTROLLER USER INTERFACE FOR A CATHETER LAB INTRAVASCULAR ULTRASOUND SYSTEM;” and U.S. Publication No. 2012/0330156 to Brown et al., published Dec. 27, 2012, titled “PULMONARY EMBOLISM THERAPEUTIC METHODS USING THERAPEUTIC ABLATION DEVICES AND SYSTEMS,” each of which is herein incorporated by reference in its entirety.

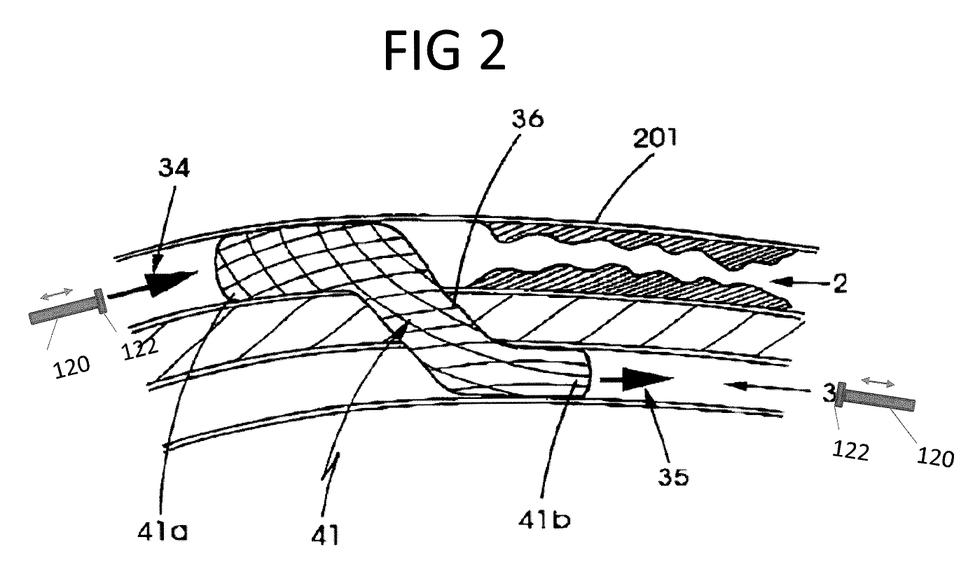

FIG. 2 is a side view of an arterial bypass stent 41 placed through tissue track 36 such that a stent proximal portion 41a is in communication with a coronary artery and a stent distal portion 41b is in communication with a coronary vein. The stent 41 may be any of a wide variety of stent or stent graft suited for bypass procedures. In some embodiments, a stent 41 is covered by a material, a dense mesh or a matrix of cells, such that coronary flow 34 cannot easily flow through the side wall of stent 41 towards stenosis 201, but instead is rerouted through stent 41 into cardiac vein 3 to produce retrograde cardiac venous flow 35. In this figure, the position of the stent suggests that a guide catheter had been placed within the coronary artery 2, and the tissue track 36 was created in the arterial to venous direction. This would allow for the proper positioning of a guidewire and subsequently the stent to allow for the device to be oriented in the arterial to venous direction. It should be clear that it is also possible for a similar stent to be placed downstream in a location, for example, corresponding to a region beyond the stenosis 201 accessed initially through vein 3 from the venous to arterial direction to permit a complete bypass of the stenosis 201 in the coronary artery 2. Stent 41 must have the capability of being dimensioned such that proximal portion 41a and distal portion 41b may be expanded into shape which closely approximates the respective wall of the vessel into which it is placed. Alternatively, a stent may be placed such that proximal portion and distal portion do not block flow, but simply act to maintain the dimensions of a tissue track or lumen.

Stents or grafts used as shown in FIG. 2 may be necessary to control dimensions of the tissue tract 36 from expanding under pressure, or closing as a result of restenosis. Another method of maintaining the dimensions of tissue track 36 permanently or temporarily during the healing and remodeling process involves use of a resorb able or biodegradable stent or stent portion used to model tissue formation of the bypassed vasculature or desirous fistula formation or geometry, as the case may be.

Still further with regard to FIG. 2, digitized data is provided to the characterization application 132 (e.g., via the IVUS catheter 120) where the digitized data corresponds to the physiology of the patient's lumen and adjacent vascular graft implantation, graft status if degradable and healthy tissue and tissue formed by healing response or as induced by graft implantation or presence. It is to be appreciated that the data may be collected from blood vessels adjacent, proximate to or joined to one or more portions of a vascular graft 36 including the tissues within tissue track 36. Still further, it is to be appreciated that the transducer 122 and/or catheter 120 may be advanced by way of example and not limitation, (i) from an artery through a graft to another artery or the same artery; (ii) from an artery through a graft to a vein as indicated by the flow arrow 34; (iii) from a vein through a graft to the same or another vein; (iv) from a vein through a graft to an artery (as would be the case if the catheter/transducer were advanced according to the indicated arrow 3). In any of the above the transducer and/or catheter may advance with or against the flow in an artery or vein or in a direction towards the heart or in a direction away from the heart or in a direction with the prevailing or intended flow or in a direction against the prevailing or intended flow.

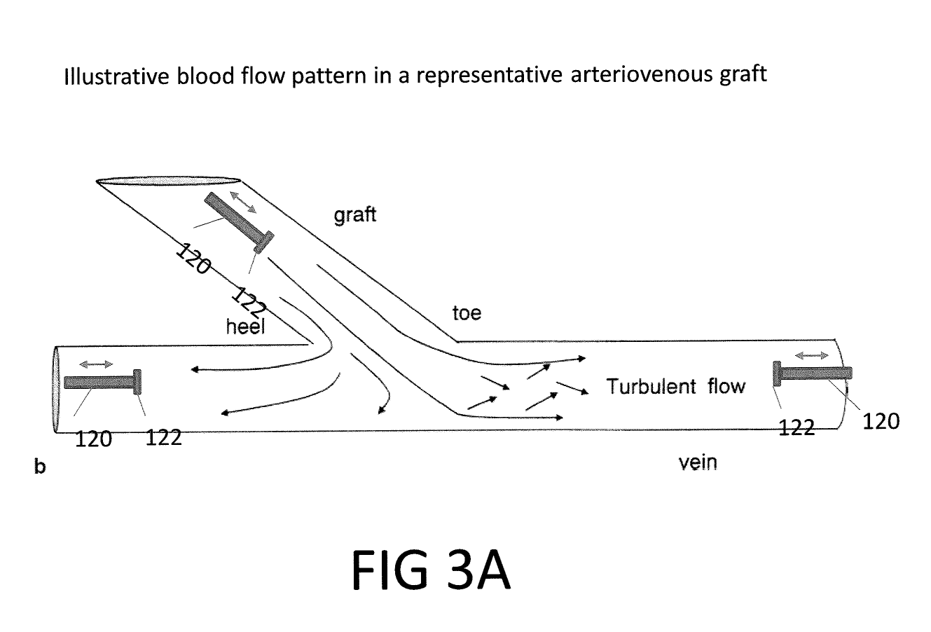

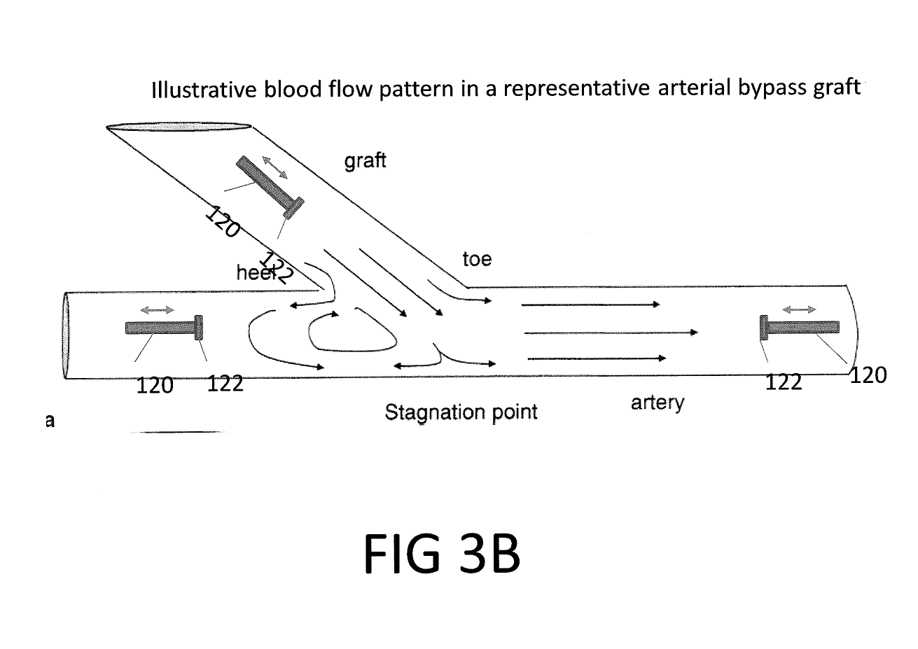

FIGS. 3A and 3B provide illustrative hemodynamic patterns within exemplary grafts depending upon graft location such as artery—vein (FIG. 3A) and artery—artery (FIG. 3B). FIG. 3A illustrates exemplary blood flow patterns at the graft/stent—vein intersection. FIG. 3B illustrates exemplary blood flow patterns at the graft/stent—artery intersection. Intravascular ultrasound, particularly those modes best suited to obtaining and analyzing hemodynamic properties, may be utilized to obtain patient specific data regarding the hemodynamic environments in the configurations of FIGS. 2, 3A, 3B.

In accordance with an embodiments of the methods described herein, digitized data is provided to the characterization application 132 (e.g., via the IVUS catheter 120) where the digitized data corresponds to the physiology of the patient's vein or artery and adjacent vascular graft implantation, graft status if degradable and healthy tissue and tissue formed by healing response or as induced by graft implantation or presence. It is to be appreciated that the data may be collected from blood vessels adjacent, proximate to or joined to one or more portions of a vascular graft including the tissues within adjacent to the graft. Still further, it is to be appreciated that the transducer 122 and/or catheter 120 may be advanced by way of example and not limitation, (i) from an artery through a graft to another artery or the same artery (as in FIG. 3B) with additional data collection near heel, toe and graft—artery hemodynamics and along the graft hemodynamics; (ii) from an artery through a graft to a vein as in FIG. 3A with additional data collection near heel, toe and graft—vein hemodynamics and along the graft hemodynamics; (iii) from a vein through a graft to the same or another vein; (iv) from a vein through a graft to an artery (as would be the case if the catheter/transducer were advanced according to the indicated arrow 3 in FIG. 2). In any of the above the transducer and/or catheter may advance with or against the flow in an artery or vein or in a direction towards the heart or in a direction away from the heart or in a direction with the prevailing or intended flow or in a direction against the prevailing or intended flow. In one aspect, the hemodynamic information is collected with particular interest in the regions indicated for turbulent flow (FIG. 3A), stagnation point (FIG. 3B) or other areas where there is increased hemodynamic stresses or forces that may impair the growth or patency of the graft or a resulting fistula, as depends on the specific implementation.

The techniques described herein may be used to identify or delineate for additional evaluation one or more boundary conditions identified by the system when the catheter 120/transducer 122 is passed through the lumen and the graft. The IVUS signals are used to penetrate into the tissue surrounding the blood vessel and discriminate between tissues of the lumen, the stent and, if applicable the growth of tissue and as needed the size, composition, location and other characteristics of the healthy tissue, tissue in the boundary condition and/or one or more properties of the graft or stent. There may be a number of boundary conditions representing an area of ultrasound image processing to evaluate, determine or estimate the hemodynamic properties within or adjacent to a transitional area such between the graft, the surrounding healthy tissue and/or new tissue growth (as in the evaluation of a fistula growth or maturation or diminution). Within a boundary condition region the rate, resolution or other factors relating to the fidelity or detail of the ultrasound image may be charged to improve details or resolution of one or more characteristics being evaluated by the system described herein. It is to be appreciated that these transitional areas may be evaluated within volumes of tissue using three dimensional ultrasound returns to identify not only the locations of or demarcations in tissue types but also the characteristics and qualities of the tissue. In still further aspects, virtual models or actual models of a graft or stent may be used to evaluate the deployment status of or changes to the implanted state of a stent or graft. In some embodiments, a stent or graft may include one or more struts, rings or other curvilinear features provided to allow the stent or graft to deploy into a desired implanted state when in use within the body. Embodiments of the present invention may be employed to also obtain IVUS information related to the status or deployment or configuration of one or more elements in a stent or graft in order to evaluate the deployment or current status of a stent or graft. In some aspects, a stent or graft may be modified or may include materials selected to enhance the ability to image the stent or graft or portion thereof using IVUS, OCT or other imaging modality.

Thereafter, the characterization application 132 uses data from the database 134 to characterize tissue corresponding to the digitized data from the IVUS catheter 120 and create a VH image (i.e., a digital image or outline that substantially corresponds to the vascular object and where specific tissue types are identified). A region of interest (ROI) on the VH image can then be identified by the operator. One region of interest (ROI) is based in whole or in part on healthy tissue surrounding a lumen or a blood vessel, graft or newly formed tissue surrounding a lumen or a blood vessel or graft or stent, and/or the size, composition, location and other characteristics of the healthy tissue, fistula tissue in the boundary condition and/or graft and/or adjacent tissue. Preferably, the ROI is characterized by the characterization data, as previously provided or obtained from other patients, the same patient or a patient having the same or similar diagnosis or course of treatment, and may be the entire VH image, hemodynamic information or a portion thereof. The characterization application 132 is then adapted to identify a corresponding region (e.g., x,y coordinates or other appropriate coordinates, etc.) on the IVUS image where the corresponding region may be specifically identified (e.g., by coloring the corresponding region with a pre-determined color). In particular, the characterization application 132 is specifically programmed to discriminate between healthy or preexisting vessel tissue and new growth or fistula tissue as well as to indicate using actual data or imported model data, structural elements of all or a portion of the stent or graft under evaluation.

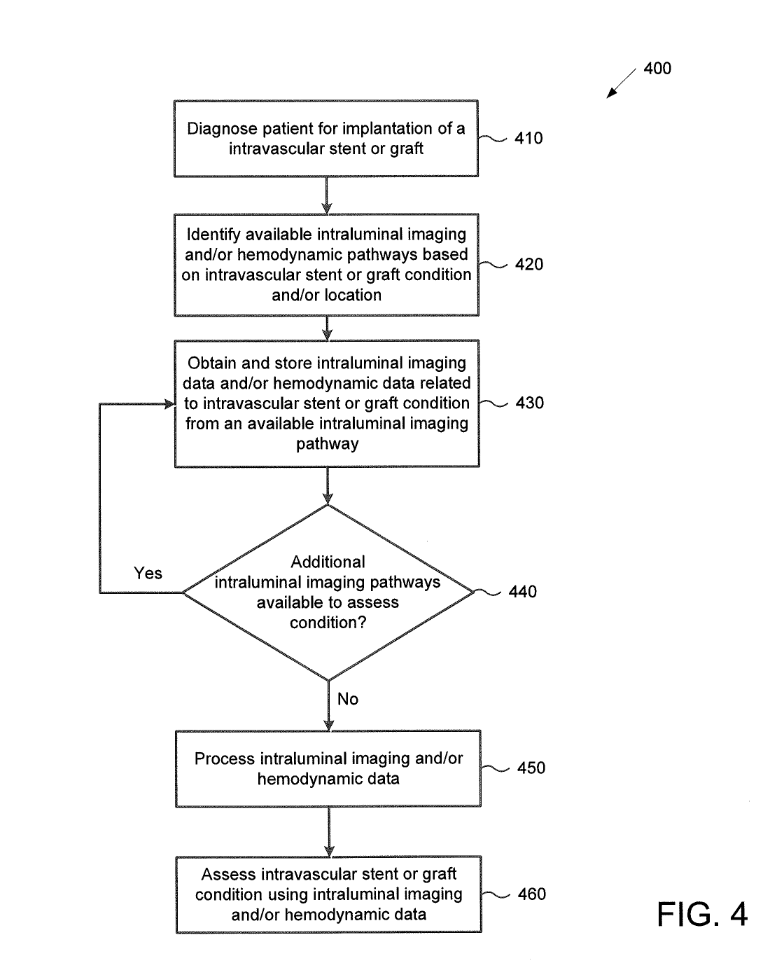

FIG. 4 illustrates a method 400 for diagnosing an intravascular stent or intravascular graft condition of a patient using, in any combination, intravascular hemodynamic information, transluminal imaging data, and/or intraluminal imaging data. The first step of diagnostic method 400 is to diagnose a patient of a condition suited for implantation of an intravascular stent or graft (step 410). Step 410 may be part of the process of checking whether a person has a disease or has an increased chance of developing a disease when the person has no symptoms. Step 410 may be related to preparing a patient for initial dialysis therapy or for continuing dialysis therapy. Next, based on the specific patient physiology and the parameters of the specific condition being treated or diagnosed, identify available intraluminal imaging pathways and or sites for hemodynamic data collection (i.e., sites of interest for intraluminal or graft-lumen evaluation, by way of example). These pathways may be via any suitable lumen of the body in order to assess the associated material, tissue or luminal conditions of the vascular graft condition being evaluated including, if appropriate, fistula formation information or indications of fistula formation impairment.

Next, at step 430, obtain and store intraluminal imaging data and/or hemodynamic data related to the lumen, stent, graft, and/or fistula condition from an available intraluminal pathway. The available intraluminal pathways including by way of example, veins, arteries, the pulmonary tree, the gut, regions of tissue surrounding the spinal column and the vessels of lymphatic system, to name a few. Next, assess whether these are additional intraluminal imaging pathways available to assess the condition (step 440). If additional pathways are available, continue to collect imaging data and/or hemodynamic or other within lumen fluid dynamic information at step 430. If all available or desired pathways have been accessed and all imaging data or hemodynamic or fluid dynamic data is collected, then the step of processing intraluminal imaging data and hemodynamic or luminal flow dynamic data is performed (step 450).

The processing step 450 includes all appropriate image processing functions in order to reconcile, orient, combine, distinguish, overlay, interpolate, transpose or otherwise relate the collected imaging data and/or hemodynamic or luminal flow information to the assessment of the vascular graft, stent or fistula condition. Finally, at step 460, there is an assessment of the vascular graft, stent or fistula condition using the intraluminal imaging data and/or hemodynamic or lumen flow dynamic information. In this step, the systems, algorithms and methods described herein draw from a wide variety of flow dynamic, hemodynamic, intraluminal imaging data including, for example, VH data or imaging data by any imaging modality from the patient being assessed or other similarly diagnosed patients or class of patients or patients undergoing the same or a different course of treatment for a stent, fistula or a vascular graft condition. While listed in serial form, for purposes of illustration the steps of processing 450 and assessing 460 as well as displaying results to a user, may be performed in a different order or in real time during the obtaining step 430.

In addition or alternatively, the VH database and/or Virtual Histology classification trees, hemodynamic data, luminal flow dynamic information also includes correlation and comparison data for transluminal ultrasound of: outer stent or graft imaging data including images or data from external imaging modalities (i.e., extracorporeal imaging such as x-ray, ultrasound, MRI, CT and the like), along with other information described herein such as location of collected image or fluid dynamic information with or without co-registration to another imaging modality, correlation of collected images to just collected/processed boundary condition information, correlation of collected images to previously collected/processed boundary condition information, each of these additional images and processing results may be from the same patient, a different general population patient or a differently situated patient.

While described above with regard to certain exemplary embodiments, it is to be appreciated that the systems and methods described herein may be applied to a wide array of vascular graft situations, disease states, treatment situations, patient types, treatment modalities (i.e., internal, external, and/or pharmacological in any combination) and other such variations. In addition or alternatively, embodiments of the systems and methods described herein may be used to advantage for pre-surgical planning, especially in the identifying the boundaries of the stent or graft before, during or after a procedure or for placement positioning, guidance, or confirmation of deployment and implanted configuration. In addition or alternatively, embodiments of the systems and methods described herein may also be adapted and configured to characterize growth or structural changes in healthy tissue adjacent to a stent or graft, within a stent or graft or supported by a stent or graft that may be undergoing absorption. In one specific aspect, the systems of methods described herein are used to evaluate or monitor the growth or development of a fistula. In another aspect, the systems and devices described herein are suited to collecting, processing, and evaluating transluminal ultrasound information obtained from any suitable lumen within the range and resolution of the imaging device for the purpose of evaluating, diagnosing, monitoring and/or aiding in the treatment of a vascular graft or fistula or a related disease state. As such, the VH database and/or Virtual Histology classification trees and/or hemodynamic or flow dynamic date used in embodiments of the systems and methods described herein also include transluminal ultrasound, imaging, correlation, comparison and/or processed data for any of a wide variety of stents, stent grafts or fistulas formed in an artery, a vein, or a lumen of a human or mammal.

In still another further aspect, embodiments of the systems and methods described herein may also be used as part of image guidance or treatment confirmation in pre-surgical, during surgical or post-surgical assessment or confirmation. In one aspect, the systems or methods herein are used for guidance of stents or grafts (with or without echogenic enhancements to improve IVUS use or being made from materials suited for improved appearance under IVUS) before, during or after a stent or graft implantation procedure or for evaluation of fistula formation. In one aspect, a portion of a stent or graft is modified to provide one or more unique or identifiable echogenic markers to aid in the evaluation or one or more characteristics of position, movement or orientation of a stent or graft, growth or development of all or a portion of a fistula or amount of absorption of an absorbable stent or graft. One or more echogenic markers or enhancements may be provided to a portion of a stent or graft according to the various techniques and materials described in International Patent Application No. PCT/US2014/027259, filed Mar. 14, 2014, titled “FILTERS WITH ECHOGENIC CHARACTERISTICS,” and International Patent Application No. PCT/US2014/027083, filed Mar. 14, 2014, titled “ENDOLUMINAL FILTER HAVING ENHANCED ECHOGENIC PROPERTIES,” each of which are herein incorporated by reference in it is entirety.

In still other aspects, embodiments of the methods and systems described herein find uses for VH, IVUS obtained hemodynamic information and IVUS imaging for identification and evaluation and/or monitoring of various forms of tissue growth as well as providing additional information useful in evaluating the local extent of fistula formation and monitoring changes in a patient or one or more patient vascular graft conditions. In one exemplary embodiment, the system and methods described herein may be used or co-register with other imaging modalities or be used to collect additional intraluminal or intravascular ultrasound data and images of possible growth or site of vascular graft investigation or therapy, or fistula formation. In addition or alternatively, this includes providing evaluation information to or receiving information from a health care provider in support of providing a patient with all of the information necessary to make fully informed treatment decisions. In addition to ultrasound imaging information there may also be information collected from other exemplary imaging modality includes for example high-resolution magnetic resonance imaging (MRI), CT scan and external imaging ultrasound.

In a still further aspect, the system and methods described herein may be used to determine, confirm the placement of and/or monitor the effectiveness of one or more vascular grafts, devices or components implanted within a lumen of the patient. The systems and methods described herein may also be used as an adjunct for the positioning, monitoring or evaluation of other luminal implants where suitable ultrasound information of the lumen and corresponding lumen flow dynamics are obtain and used as described elsewhere herein.

In a still further aspect, the system and methods described herein may be used for evaluation of the effectiveness of a drug treatments for fistula formation, or tissue growth in or around or within a graft or to provide information and/to assess the size, shape, density or other characteristics to determine changes in size as well as provide identification of fistula or graft tissue forming or progression thereof.

In still another aspect, there may also be collected in or available to the characterization application 130 information about one or more indicia of or characteristics of one or more biomarkers found in or correlated to one or more samples in the VH or indications database—if a type of tissue or vascular graft implantation or fistula formation composition correlates to the presence of, amount of or absence of a biomarker. A biomarker is a biological molecule found in blood, other body fluids, or tissues that is a sign of a normal or abnormal process, or of a condition or disease. A biomarker may be used to see how well the body responds to a treatment for a disease or condition. A biomarker may also be a molecular marker or a signature molecule. In addition or alternatively, any other IVUS or imaging detectable indicia that may distinguish healthy tissue, lumen wall, graft wall, fistula or other tissue as may be useful in the methods and systems described herein.

In still another aspect, the systems and methods described herein may be used in providing additional information useful in evaluating and monitoring changes along with capabilities to use or co-register with other imaging modalities or be used to collect additional intraluminal and/or intravascular ultrasound images of possible growth or site of investigation or vascular graft implantation, fistula growth or an associated therapy, such as dialysis in one example.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for assessing a hemodynamic condition in a patient having an implanted vascular graft, comprising:
Advancing an IVUS capable catheter through a lumen within or adjacent to the implanted vascular graft or a portion of the vascular graft;
Collecting intraluminal ultrasound information related to one or more hemodynamic characteristics of a flow within the lumen or the portion of the vascular graft; and
Comparing the information from the collecting step to information related to a desired hemodynamic characteristic of a lumen portion within or adjacent to an implanted vascular graft or within a portion of an implanted vascular graft using an image comparison algorithm; and
Providing a result of the comparing step.

2. The method of claim 1 wherein the collecting step is performed periodically after implantation of the vascular graft.

3. The method of claim 1 wherein the vascular graft is an arterial bypass graft.

4. The method of claim 1 wherein the vascular graft is an arteriovenous graft.

5. The method of claim 3 wherein the steps of advancing and collecting are performed in a blood vessel adjacent, proximate to or joined to one or more portions of the arterial bypass graft.

6. The method of claim 4 wherein the steps of advancing and collecting are performed in a blood vessel adjacent, proximate to or joined to one or more portions of the arteriovenous graft.

7. The method of claim 1 wherein the one or more hemodynamic characteristics include one or more of wall shear stress, an indication of turbulent flow, an indication of laminar flow, an indication of blood velocity, and an indication of one or more blood velocity patterns.

8. The method of claim 1 wherein when the advancing and the collecting step are performed a transducer or a catheter is moved from one or more of from an artery through a graft to another artery or the same artery; (ii) from an artery through a graft to a vein; (iii) from a vein through a graft to the same or another vein; (iv) from a vein through a graft to an artery.

9. The method of claim 1 further comprising: moving an IVUS transducer or catheter with or against the flow in an artery or vein or in a direction towards the heart or in a direction away from the heart or in a direction with the prevailing or intended flow or in a direction against the prevailing or intended flow.

10. A method for assessing a condition of a tissue within or adjacent to an implanted vascular graft, comprising:
Advancing an IVUS capable catheter through a vascular lumen within or adjacent the implanted vascular graft;
Collecting intraluminal ultrasound information related to a composition of a portion of the vascular lumen or vascular graft; and
Comparing the information from the collecting step to information in a virtual histology tree related to the composition of the portion of the vascular lumen or the vascular graft using an image comparison algorithm; and
Providing a result of the comparing step.

11. The method of claim 10 wherein the collecting step is performed after a time period has elapsed after implantation of the vascular graft.

12. The method of claim 10 wherein the collecting step is performed periodically after implantation of the vascular graft.

13. The method of claim 10 wherein the vascular graft is an arterial bypass graft.

14. The method of claim 10 wherein the vascular graft is an arteriovenous graft.

15. The method of claim 10 wherein the steps of advancing and collecting are performed in a blood vessel adjacent, proximate to or joined to one or more portions of the arterial bypass graft.

16. The method of claim 10 wherein the steps of advancing and collecting are performed in a blood vessel adjacent, proximate to or joined to one or more portions of the arteriovenous graft.

17. The method of claim 10 wherein the steps of advancing and collecting are performed in a blood vessel adjacent, proximate to, along or within a fistula.

18. The method of claim 10 wherein the steps of advancing and collecting are performed in a blood vessel adjacent, proximate to, along or within a fistula so as to obtain one or more hemodynamic characteristics including one or more of wall shear stress, an indication of turbulent flow, an indication of laminar flow, an indication of blood velocity, and an indication of one or more blood velocity patterns.

19. The method of claim 10 wherein when the advancing and the collecting step are performed a transducer or a catheter is moved from one or more of from an artery through a graft to another artery or the same artery; (ii) from an artery through a graft to a vein; (iii) from a vein through a graft to the same or another vein; (iv) from a vein through a graft to an artery.

20. The method of claim 10 further comprising: moving an IVUS transducer or catheter with or against the flow in an artery or vein or in a direction towards the heart or in a direction away from the heart or in a direction with the prevailing or intended flow or in a direction against the prevailing or intended flow.

21. A method for assessing a vascular graft condition in a patient, comprising:
Advancing an IVUS capable catheter through a lumen within or adjacent a site of an vascular graft condition in a patient;
Collecting transluminal or intraluminal ultrasound information on the vascular graft or other lesion or condition of the patient; and
Comparing the information from the collecting step to information in a virtual histology tree related to the vascular graft condition using an image comparison algorithm; and
Providing a result of the comparing step to a health care provider.

22. The method of claim 21 wherein the collecting step is performed after the patient has received one or more treatments for a vascular graft condition.

23. The method of claim 21 wherein the providing the result includes relative information about the size, composition, or position of a fistula or region of interest related to the vascular graft condition.

24. The method of claim 21 further comprising: collecting ultrasound information of a portion of a stent or graft to determine the location, position, orientation, configuration or amount or degree of deployment or one or more portions, components or elements of the stent or graft.

25. The method of claim 21 wherein the step of advancing an ultrasound probe or catheter is performed with particular regard to one or more regions of interest for the configuration of the stent-graft under evaluation.

26. The method of claim 1 further comprising: collecting ultrasound information of a portion of a stent or graft to determine the location, position, orientation, configuration or amount or degree of deployment or one or more portions, components or elements of the stent or graft.

27. The method of claim 1 wherein the step of advancing an ultrasound probe or catheter is performed with particular regard to one or more regions of interest for the configuration of the stent-graft under evaluation.

28. The method of claim 10 further comprising: collecting ultrasound information of a portion of a stent or graft to determine the location, position, orientation, configuration or amount or degree of deployment or one or more portions, components or elements of the stent or graft.

29. The method of claim 10 wherein the step of advancing an ultrasound probe or catheter is performed with particular regard to one or more regions of interest for the configuration of the stent-graft under evaluation.
